(19) United States
(12) Patent Application Publication
(10) Pub. No.: US 2022/0395310 A1
(43) Pub. Date: Dec. 15, 2022

(54) FREQUENCY BASED CONTROLLED ELECTROSURGICAL SYSTEM AND METHOD

(71) Applicant: Intuitive Surgical Operations, Inc., Sunnyvale, CA (US)

(72) Inventors: Duane W. Marion, Scottsdale, AZ (US); Andrew D. Rauch, San Francisco, CA (US); Jignesh M. Shah, Sunnyvale, CA (US)

(21) Appl. No.: 17/834,898

(22) Filed: Jun. 7, 2022

Related U.S. Application Data
(60) Provisional application No. 63/209,751, filed on Jun. 11, 2021.

(51) Int. Cl. A61B 18/12

(52) U.S. Cl. CPC A61B 18/12; A61B 2018/00857

(57) ABSTRACT

A method is provided to control delivery of heat to biological tissue comprising: imparting an RF electrical signal to the biological tissue electrically coupled between a first electrode and the second electrode; measuring frequency content of the RF electrical signal between the first electrode and the second electrode; and adjusting the RF electrical signal based upon the measured frequency content of the RF electrical signal.

### CLAIM OF PRIORITY

This application claims the benefit of priority under 35 U.S.C. § 119(e) to U.S. Patent Application Ser. No. 63/209,751, filed on Jun. 11, 2021, which is incorporated by reference herein in its entirety.

### BACKGROUND

Electrosurgery involves the use of electricity to buildup heat within biological tissue to cause thermal tissue damage through one or more of desiccation, coagulation, or vaporization, resulting in incision, removal or sealing of the tissue. High frequency electrosurgery involves heating biological tissue by imparting radio frequency (RF) alternating current (AC) to biological tissue such that the current is converted to heat by resistance as it passes through the tissue. Benefits include the ability to make precise incisions with limited blood loss during surgical procedures in hospital operating rooms or in outpatient procedures.

An electrosurgical instrument creates a relatively high voltage potential between an active electrode and the target tissue. This high voltage quickly dessicates the tissues and in many cases, creates a vapor barrier between the electrode and the tissue surface. With a high enough voltage, an electrical plasma is created in this vapor barrier, and the ionized elements of this plasma allow RF current conduction from the electrode to the tissue, which causes vaporization of the target tissue that results in mechanical separation or dissection.

Electrosurgery can be performed with either a monopolar or a bipolar electrosurgical instrument each of which includes two electrodes to contact biological tissue. In some cases, an active electrode can make contact with non-target conductive surfaces such as other instruments, staples or fluids such as blood or saline, which can result in a sudden breakdown in impedance across the vapor barrier. The sudden reduced impedance can result in an electrical arc at the active electrode or can result in an electrical arc between the active electrode and the return electrodes of a bipolar device. An arc event can cause excessive tissue damage or damage to the electrodes themselves.

Thus, there is a need to rapidly detect an onset of an arc event and to control the application of RF current to avoid occurrence of the arc event or to reduce intensity of the arc event.

### SUMMARY

In one aspect, a method to control delivery of heat to biological tissue. An RF electrical signal is imparted to the biological tissue over a circuit that includes a first electrode and a second electrode, wherein the biological tissue is electrically coupled between the first electrode and the second electrode. Frequency content of the RF electrical signal is measured. The RF electrical signal is adjusted based upon the measured frequency content of the RF electrical signal.

In another aspect, an electrosurgical system is provided to deliver heat to biological tissue. A radio frequency (RF) electrical signal source is operable to impart an RF electrical signal to the biological tissue. A digital sampling circuit is coupled to digitally sample the RF electrical signal imparted to the biological tissue. Frequency content logic is coupled to determine frequency content of the RF electrical signal based upon digital samples of the RF electrical signal. Control logic is coupled to control the RF electrical signal source based upon the determined frequency content of the RF electrical signal.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 is an illustrative example system level block diagram representing setup of an electrosurgical system.

FIG. 2 is an illustrative drawing showing an example RF voltage signal component and an example RF current signal component of an RF electrical signal imparted to an example biological tissue load in the absence of an aberrant arcing event.

FIG. 3 is an illustrative drawing showing an example RF voltage signal component and an example RF current signal component of an RF electrical signal imparted to an example biological tissue load during an occurrence of an aberrant arcing event.

FIG. 4 is an illustrative block diagram representing an example electrosurgical signal generator circuit.

FIG. 5A is an illustrative perspective view of a pair of example jaws of an end effector that include a set of tissue sealing surfaces and a set of tissue cutting surfaces shown in an open position.

FIG. 5B is a distal end view of the pair of example jaws of FIG. 5A shown in a closed position with biological tissue grasped between them.

FIG. 6 is an illustrative signal timing diagram showing example peak-to-peak current signal levels of simultaneous sealing and cutting signals in accordance with some embodiments.

FIG. 7 is an illustrative flow diagram representing a process to control an electrosurgical signal generator based upon frequency content of an electrical signal across a biological tissue electrical load.

FIG. 8 is an illustrative graph that shows a comparison between an FFT of a waveform influenced by an arc event arc and a wavefrom not influenced by an arc event.

FIG. 9 is an illustrative flow diagram representing certain details of an adjustment operation.

### DETAILED DESCRIPTION

A. Overview

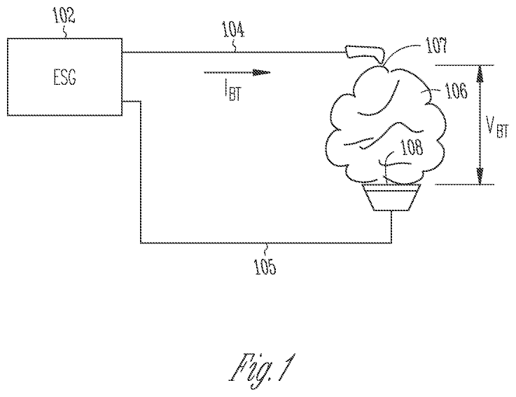

FIG. 1 is an illustrative example system level block diagram representing system setup of an electrosurgical signal generator (ESG) 102 to impart RF energy to biological tissue 106. The ESG 102 is electrically coupled to first conductor 104 and a second conductor 105. During a medical procedure, a patient's biological tissue 106 is electrically coupled between the first conductor 104 and a second conductor 105. The first conductor 104 electrically contacts a first location 107 on the tissue portion 106 and the second conductor 105 electrically contacts a second location 108 on the tissue portion 106 to create an electrical circuit that includes the generator 102, the first and second conductors 104, 105 and the tissue portion 106. The electrosurgical signal generator 102 delivers an RF electrical signal to the circuit in which the tissue portion 106 acts as an electrically resistive load during non-arc conditions. During a medical procedure, the generator 102 generates an RF current that causes a current I_{BT} to flow through the biological tissue portion and that causes an RF voltage across V_{BT} across the biological tissue. An example electrosurgical system 100 can be configured as a monopolar system in which the first conductor 104 includes an active electrode and the second conductor 105 includes a return electrode pad (not shown) that is attached to the patient. An alternative example ESG 102 can be configured as a bipolar system in which the first conductors 104, 105 are electrically coupled to opposed jaws (not shown) of forceps such that current flows through the biological tissue 106 located between the jaws.

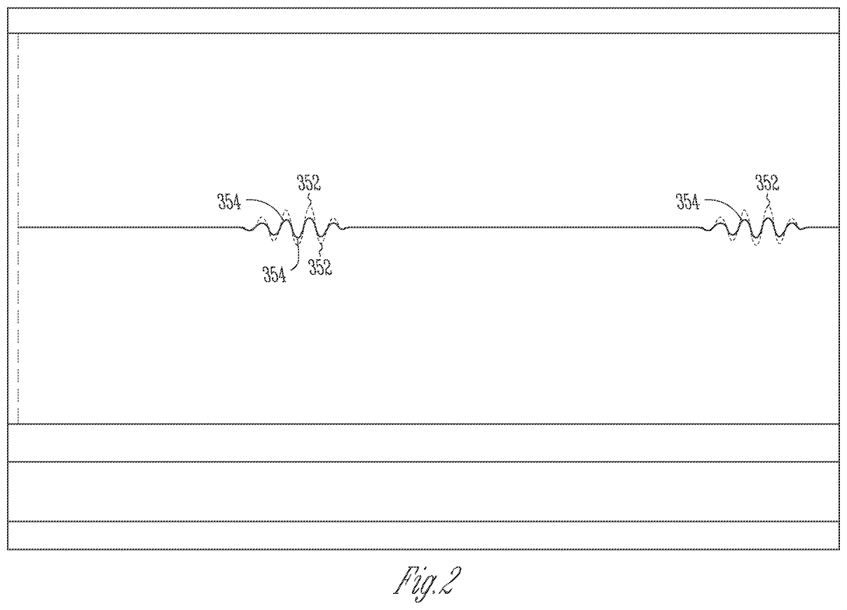

FIG. 2 is an illustrative drawing showing an example RF voltage signal component 352 and an example RF current signal component 354 of an RF electrical signal imparted to an example 100-ohm biological tissue load in the absence of an aberrant arcing event. The electrosurgical generator 102 delivers an RF electrical signal to deliver RF energy to the tissue portion 106 over the first and second conductors 104, 105. In general, biological tissue 106 is primarily electrically resistive. When an alternating current waveform is applied to a biological tissue load, in the absence of an arc event, the load behaves linearly, and the voltage and current waveforms have substantially the same shape. In the example waveforms shown in FIG. 2, if a sinusoidal voltage waveform is applied to a linear biological tissue load, then the resultant current delivered to the load is also a sinusoidal waveform. The frequency components of the current and voltage waveforms across the biological tissue substantially match one another and match the voltage and current components of the RF sinusoidal signal delivered by the generator 102. It is noted that there may be some mismatching due to an acceptable amount of low-level arcing within plasma formed at tissue locations where a conductor contacts the tissue.

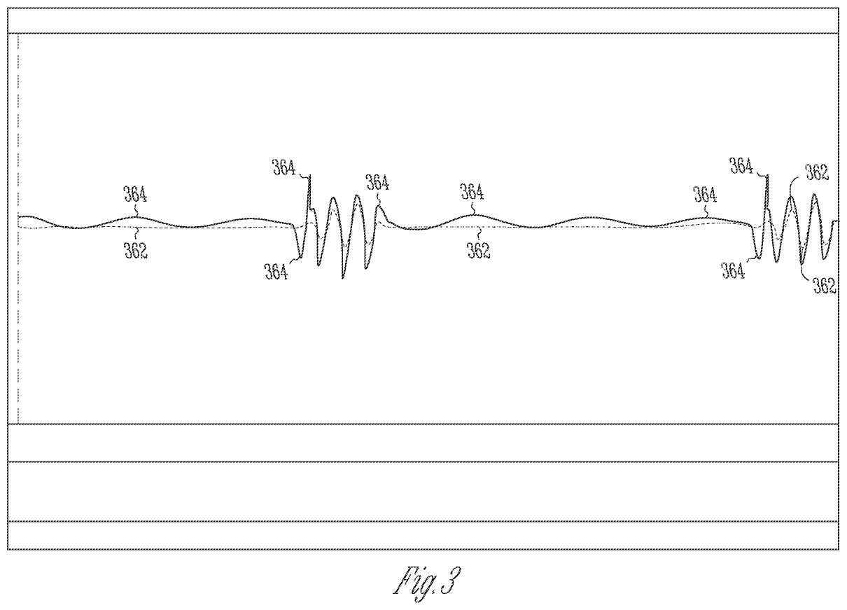

FIG. 3 is an illustrative drawing showing an example RF voltage signal component 362 and an example RF current signal component 364 of an RF electrical signal imparted to an example 100-ohm biological tissue load during an occurrence of an aberrant arcing event. An aberrant arc event is an arcing event that can cause unwanted damage to biological tissue or to a medical instruments or equipment. During an aberrant arcing event, an electrical arc discharging through a conductive plasma introduces unacceptable non-linear effects to the load. Assuming that the generator 102 generates a sinusoidal signal like that shown in FIG. 2, an aberrant arc event at the biological tissue load can cause the shape of the waveform to diverge from that of the voltage waveform. In the example waveforms shown in FIG. 3, during an aberrant arc event, the current and voltage across the biological tissue load have differing high frequency components that do not match. A distortion in the waveforms is brought about by the introduction of high frequency elements to the AC current waveform that occur as a result of the discharge. This non-linear response is a result of the cascade effect introduced by the electrode motion that results in current flow through a plasma. As the electrons move, they collide with non-ionized gas molecules, which can result in electrons being freed from their molecular orbits, thereby creating more free electrons which can be used for current delivery. During an aberrant arc event, the result is a significant difference in amounts of distortion of the current and voltage waveforms. In general, the current waveform is much more distorted due to an aberrant arc event than is the voltage waveform.

Thus, during electrosurgery, the frequency content of the current flow through target biological tissue and the voltage across the target biological tissue are indicative of the severity of arc event. A clinically minor arc event often occurs in the normal course of a medical procedure and can contribute to a successful outcome. However, a clinically unacceptable more severe arc event can occur that, if not properly managed, can have deleterious consequences. As illustrated in FIGS. 2-3, comparative frequency content of acurrent flow through target biological tissue portion and the voltage across the target biological tissue portion is indicative of an arcing event.

A system and method are provided to distinguish between a clinically acceptable minor arc event and a clinically unacceptably more severe arc event. By comparing frequency content of an RF voltage imparted to the biological tissue with frequency content of a corresponding RF current imparted to the biological tissue, a determination is made whether an aberrant arc event is occurring or is about to occur. RF energy delivery by the generator 102 is adjusted based upon the comparison. For example, a voltage level at which an RF current signal is delivered to tissue is reduced to reduce RF energy delivery to the tissue, and conversely, a voltage level at which an RF current signal is delivered to tissue is increased to increase RF energy delivery to the tissue. In response to a difference in frequency content of the current and voltage components that is within a clinically acceptable threshold limit, an example generator 102 is caused to continue generating RF energy delivery according to a predetermined clinical procedure protocol. In response to a difference in frequency content of the current and voltage components that exceeds the clinically acceptable threshold limit, the example generator 102 is caused to adjust RF energy delivery to stop or to limit the aberrant arc event.

B. Electrosurgical Signal Generator

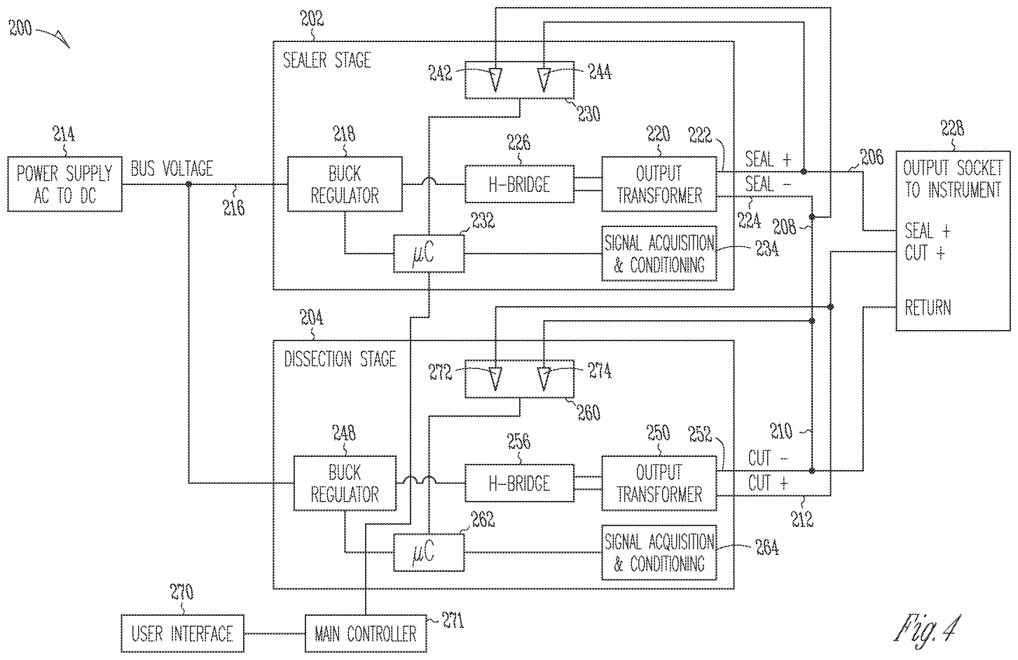

FIG. 4 is an illustrative block diagram representing an example electrosurgical signal generator (ESG) 200. The ESG 200 includes an electrosurgical signal generator sealing stage 202 and an ESG cutting stage 204. The sealing stage 202 produces a high frequency (HF) AC sealing signal between a set of sealing electrodes 206, 208 (seal+, seal−). The cutting stage 204 produces a HF AC cutting signal between a set of cutting electrodes 210, 212 (cut+, cut−). Typically, the frequency is in a range of approximately 100-100 kHz. In the example ESG 200, the set of sealing stage electrodes 206, 208 and the set of cutting stage electrodes 210, 212 each shares at least one electrode (208, 212) (seal−, cut−) in common, which may be referred to collectively herein as the return electrode.

The ESG 200 includes an AC-to-DC power supply 214 to convert an AC line voltage to a DC voltage on a voltage bus line 26216. The voltage bus line 26216 is coupled to provide a DC input voltage signal to the sealing stage circuit 202. The voltage bus line 26216 also is coupled to provide the DC input voltage signal to the cutting stage circuit 204. In some embodiments, the DC input voltage signal is approximately 48V, for example.

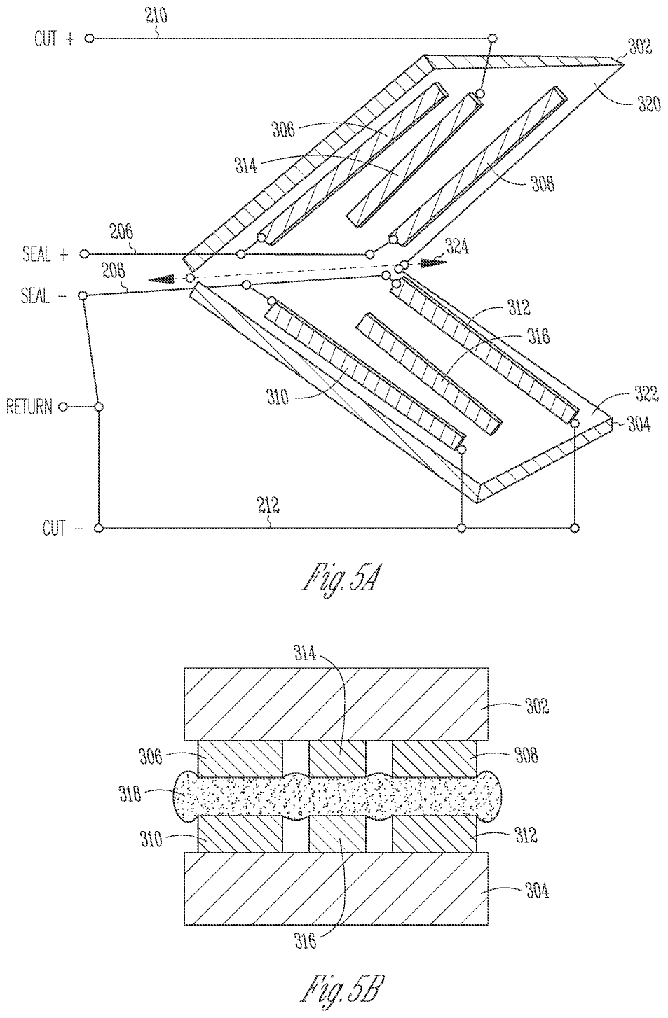

The sealing stage 202 includes a first RF signal source that includes a first buck regulator circuit 218 to convert the DC input voltage signal to a first controlled DC voltage signal and includes a first output transformer 220 coupled to produce the AC sealing signal based upon the first controlled DC voltage signal. The first output transformer 220 is coupled to provide the sealing signal to the set of sealing electrodes 206, 208. More particularly, the first output transformer 220 includes a first output terminal 222 electrically coupled to the first sealing electrode 206 and includes a second output terminal 224 electrically coupled to the second sealing electrode 208. In some embodiments, the first output stage includes a first H-bridge switch circuit. The first and second sealing electrodes 206, 208 are electrically coupled via an output socket 228 to opposed first and second jaws 303, 304 described below with reference to FIGS. 5A-5B. Thus, the first RF signal source is operable to impart an RF electrical signal over a circuit that includes the first output terminal 222 electrically coupled to the first sealing electrode 206 and that includes a second output terminal 224 electrically coupled to the second sealing electrode 208, with biological tissue electrically coupled between the first and second sealing electrodes 206, 208. A first voltage and current monitoring sensor circuit 230 includes first and second analog-to-digital converter circuits 242, 244 configured to respectively monitor, through digital sampling, sealer stage current and sealer stage voltage across the set of sealing electrodes. A first micro-controller 232 is configured to provide a pulse width modulated (PWM) signal to the first buck regulator circuit 218 to determine the voltage conversion imparted by the first buck regulator circuit 218. The first micro-controller 232 also is configured to produce a control signal to control switching of the output stage switch circuit 226 to thereby determine the RF sealing signal waveform pattern, including duty cycle and frequency, for example. An example first micro-controller 232 includes a processor circuit that includes logic circuitry that is dynamically configured based upon program instructions to perform operations. An alternate example first micro-controller 232 includes a Field Programmable Gate Array (FPGA) circuit that includes pre-programmed logic circuits. First signal conditioning and acquisition circuitry 234 acquires the voltage and current measurements that can be used to calculate voltage and current across biological tissue electrical load electrically coupled between the jaws 302, 304.

Similarly, the cutting stage 204 includes second RF signal source that includes a second buck regulator circuit 248 to convert the DC input voltage signal to a second controlled DC voltage signal and includes a second output transformer 250 coupled to produce the cutting signal based upon the second controlled DC voltage signal. The second output transformer 250 is coupled to provide the AC cutting signal to the set of cutting electrodes 210, 212. More specifically, the second output transformer 250 includes a first output terminal 252 electrically coupled to the first cutting electrode 210 and includes a second output terminal 254 electrically coupled to the second cutting electrode 212. The set of cutting electrodes 210, 212 are electrically coupled via the output socket 228 to opposed first and second jaws 303, 304 described below with reference to FIGS. 5A-5B. Thus, the second RF signal source is operable to impart an RF electrical signal over a circuit that includes the first output terminal 252 electrically coupled to the first cutting electrode 210 and that includes a second output terminal 254 electrically coupled to the second cutting electrode 212, with biological tissue electrically coupled between the first and second cutting electrodes 210, 212. A second current and voltage monitoring sensor circuit 260 includes third and fourth analog-to-digital converter circuits 272, 274 configured to respectively monitor, through digital sampling, dissection stage current and dissection stage voltage across the set of cutting electrodes 210, 212. A second micro-controller 262 is configured to provide a pulse width modulated (PWM) signal to the second buck regulator circuit 248 to determine the voltage conversion imparted by the second buck regulator circuit 248. The second micro-controller 262 also is configured to produce a control signal to control switching of the output stage switching circuit 256 to thereby determine the RF cutting signal waveform pattern, including duty cycle and frequency, for example. An example second micro-controller 262 includes a processor circuit that includes logic circuitry that is dynamically configured based upon program instructions to perform operations. An alternate example second micro-controller 262 includes a Field Programmable Gate Array (FPGA) circuit that includes pre-programmed logic circuits. Second signal conditioning and acquisition circuitry 164 acquire the voltage and current measurements can be used to calculate voltage and current across biological tissue electrical load electrically coupled between the jaws 302, 304.

A user interface circuit (UI) block 270, is configured to receive user input commands to start and stop sealing and cutting activities and to indicate parameters to use for sealing and cutting signal waveforms such as voltage, current, signal frequency, and dwell time, for example. The UI circuit block 270 also may provide feedback information to the user such as amount of power delivered, whether a seal was successfully completed, whether an error condition occurred. A surgeon may use the UI to provide user input to select voltage and current levels or sealing signal patterns and cutting signal patterns based upon requirements of a particular patient or surgical procedure, for example. A main controller 271 is coupled to exchange information with the UI block 270 and to communicate with the first and second micro-controllers 232, 262. The main controller 271 may be configured to produce control signals to determine waveforms of the sealing and cutting signals under control of the first and second micro-controllers, including current and voltage levels, for example. The main controller 271 also may produce control signals to determine parameters such as, RF pulse duration, RF pulse repetition rate, and total number of RF pulses during a sealing or cutting procedure, which may be controlled by the first and second micro-controllers.

In operation, an AC sealing signal is provided via the first output transformer 220 across the set of sealing electrodes 206, 208, and an AC cutting signal is provided via the second output transformer 210 across the set of cutting electrodes 210, 212. In some embodiments, the first and second micro-controllers 232, 262 cooperate to provide a single PWM master signal to the first and second H-bridge switches 226, 256 to produce in-phase periodic sealing and cutting signals. Although the sealing and cutting signals are periodic signals that are in phase with each other, they typically have different peak-to-peak voltage potentials. The first and second output transformers 220, 250 may have different turn ratios to produce different voltage levels for the sealing and cutting voltages, for example. In general, impedance is lower during a sealing activity than during a cutting activity due to the higher impedance associated with the plasma discharge required to resect tissue. Thus, in general, a lower voltage ordinarily may be used during sealing than is used during a cutting. In some embodiments, for example, the peak-to-peak voltage for a sealing activity is approximately 71-210V and the peak-to-peak voltage for a cutting activity is approximately 300-600V. Conversely, in general, a higher current may be used during sealing than is used during a cutting.

FIG. 5A is an illustrative perspective view of a pair of opposed jaws 302, 304 that include a set of tissue sealing surfaces 306-312 and a set of tissue cutting surfaces 310, 312 and 314 shown in an open position in accordance with some embodiments. Thus, sealing surfaces 310, 312 are shared between the sealing stage 202 and the cutting stage 204. FIG. 5B is a distal end view of the pair of end effector jaws 302, 304 of FIG. 5A shown in a closed position with biological tissue 318 grasped between them in accordance with some embodiments. Referring to FIG. 5A, the first and second jaws 302, 304 have opposing working faces 320, 322 and a pivot axis 324. At least one of the first and second jaws 302, 304 is mounted to rotatably pivot about the pivot axis 324 between the open position in which the first and second jaws 302, 304 are spaced apart from each other and the closed position for grasping biological tissue 318 between them.

The first jaw 302 includes first and second electrically conductive tissue sealing surfaces 306, 308 that are electrically coupled at the socket 228 to the active sealing electrode 206 and that extend longitudinally along outer portions of the first jaw 302. The first jaw 302 also includes an electrically conductive tissue cutting surface 314 that is electrically coupled at the socket 228 to the active cutting electrode 210 and that extends longitudinally along the first jaw 202 between the first and second tissue sealing surfaces 306, 308. The second jaw 304 includes third and fourth electrically conductive tissue sealing surfaces 310, 312 that are electrically coupled at the socket 228 to the shared return sealing electrode 208 and that extend longitudinally along outer portions of the second jaw 204 so as to align with the first and second tissue sealing surfaces 306, 308 when the first and second jaws 302, 304 are in the closed position. The second jaw 304 also includes a passive/insulative surface 316 that extends longitudinally along the second jaw 304 between the third and fourth tissue sealing surfaces 310, 312 so as to align with the first tissue cutting surface 314 when the first and second jaws 302, 304 are in the closed position.

Referring to FIG. 5B, during tissue sealing activity, the sealing signal is conducted through tissue portion 318 disposed between the first and third sealing surfaces 206, 210 and through tissue portion 318 disposed between the second and fourth tissue sealing surfaces 308, 312. During tissue cutting, the cutting signal is conducted though a tissue portion 318 disposed between the first and second tissue cutting surfaces 310, 312, 314. Often, it is beneficial to start a sealing activity before a cutting activity for reduced clinical risk. In this way, if biological tissue, such as a blood vessel, is sealed to some pre-determined extent before cutting begins, there is minimal risk of blood leakage during a later-started cutting activity.

In general, the voltage and current density applied to a biological tissue determines whether cutting or sealing of the tissue occurs, as a higher voltage and current density is required to achieve the plasma discharge required for resection. A lower current density typically results in less rapid tissue heating, which may result in sealing, which as used herein, refers to tissue dehydration, vessel wall shrinkage and coagulation of blood constituents and collagen denaturatization and bonding. A higher current density typically results in the creation of a plasma discharge, which may result in cutting, which as used herein, refers to dissecting of tissue through vaporization, for example. Although electrosurgical sealing signals and electrosurgical cutting signals may deliver the same power, they ordinarily use different voltage and current levels to do so.

A typical electrosurgical procedure that involves both sealing and cutting activities may involve a sequence of “bites” in which a pair of jaws grasp a tissue portion, the electrosurgical generator provides sealing and cutting signals to seal it and cut it, and then a next portion of tissue is grasped, sealed and cut, etc. Each bite of sealing activity and each cutting activity may require only a short time interval, such as two seconds to seal and two seconds to cut, for example. The overall time required for an electrosurgical procedure increases with an increasing number of bites. For example, an electrosurgical procedure involving 5-6 bites in which sealing and cutting activities are performed in sequence may require 20-24 seconds. Moreover, if a single stage electrosurgical generator is used, then an additional time delay of perhaps 4-5 seconds per bite may be required, for example, to reconfigure the generator to generate a different signal pattern at each transition between a sealing and a cutting activity, which can further increase the overall time for an electrosurgical procedure by an additional 20-30 seconds, for example. Thus, there is need for simultaneous sealing and cutting to shorten the time required for an electrosurgical procedure.

C. Sealing and Cutting Signals

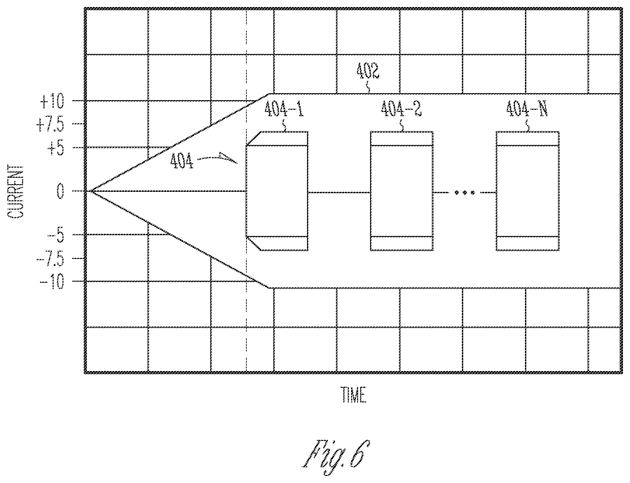

FIG. 6 is an illustrative signal timing diagram showing example peak-to-peak current signal levels of simultaneous sealing and cutting signals. The example RF peak-to-peak current sealing and cutting signals of FIG. 6 represent energy delivery according to an example predetermined clinical procedure protocol. It will be appreciated that the drawing is illustrative and current level units and time units are arbitrary and for illustrative purposes only. The example peak-to-peak current value of the RF sealing current signal 402 is greater than the example peak-to-peak current value of the RF cutting current signal pulses 404-1 to 404-N. The sealing signal 402 is provided as a continuous RF signal. Whereas, the cutting signal 404 is provided in discrete RF signal pulses 404-1 to 404-N during discrete time intervals with a dead signal dwell time delay between each pulse during which no cutting signal is provided. Each RF signal pulse includes an RF cutting signal imparted continuously during the pulse time interval. Each dead signal dwell time delay includes a time interval during which no RF cutting signal is imparted. The number of cutting pulses may vary as required to achieve a satisfactory cut based upon a measure of impedance between the set of cut electrodes 210, 212, for example. The sealing and cutting signals provide substantially the same power, and therefore, while the sealing signal current level is greater than the cutting signal current level, the sealing signal voltage level (not shown) is less than the cutting signal voltage level (not shown).

D. Adjusting RF Energy Delivery to Biological Tissue Based Upon Frequency Content of Voltage and Current Waveforms

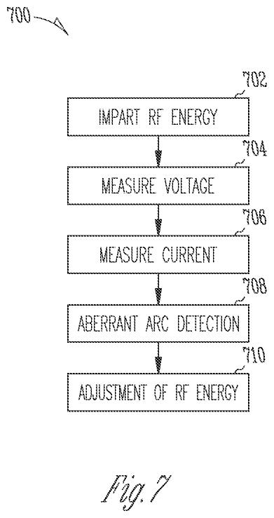

FIG. 7 is an illustrative flow diagram representing a process 700 to control an ESG based upon frequency content of an electrical signal across a target biological tissue electrical load. The example electrosurgical generator 200 of FIG. 4 includes a sealer stage 202 that uses RF energy to seal tissue and includes a dissection stage 204 that uses RF energy to cut biological tissue. As explained above, different combinations of electrodes are used during the cutting and sealing. Accordingly, the generator 200 is configured to run the process 700 during both during cutting and sealing. Logic circuitry of the first and second micro-controllers 232, 262 is configured using program instructions to implement the operations of the process 700.

During an electrosurgical sealing procedure an RF energy delivery operation 702 imparts an RF electrical signal to deliver RF energy at a predetermined clinical level to a biological tissue portion electrically coupled between first and second electrode conductors located at an electrosurgical instrument end effector. In an example ESG, a voltage measurement operation 704 continuously measures frequency content of a voltage waveform across the electrodes. In an example ESG, a current measurement operation 706 continuously measures frequency content of a current waveform at the first and second conductors. In an example ESG, an arc detection operation 708 continuously evaluates whether there is an occurrence aberrant arc event, based upon a combination of the measured frequency content of the voltage waveform and the frequency content of the current waveform. As explained below, an alternate example arc detection operation detects an occurrence of an arc event based upon measured current content. An adjustment operation 710 adjusts delivery of RF energy based upon outcome of the arc detection operation 708. Following adjustment operation 710, control next flows back to operation 702 so that the process 700 runs continuously throughout a electrosurgical procedure.

Referring to the example ESG 200 of FIG. 4, during an electrosurgical sealing procedure using the sealer stage 202, operation 702 the first microcontroller 232 causes the first buck regulator 218 to produce a voltage control signal to control the voltage level of an RF output signal provided by the first output transformer 220 according to a clinical sealing procedure protocol. The output voltage level the first output transformer 220 determines the RF energy level delivered to biological tissue 318. In the absence of an aberrant arc event, the first microcontroller 232 causes the first output transformer 220 to output an RF signal having a predetermined clinical energy level according to a predetermined clinical procedure protocol suitable to seal biological tissue. The predetermined clinical energy level includes current and voltage values selected to deliver energy to a biological tissue load coupled between the conductors 206, 208 that is sufficient to seal tissue without causing unwanted tissue damage and without causing unwanted damage to the instrument, for example. The predetermined clinical energy level may cause some acceptable level of arcing within a plasma layer that may be formed near the surface of a tissue portion to be sealed. During operation 704, the example first microcontroller 232 causes the first sensor circuit 230 to continuously measure voltage across the first and second sealing electrode conductors to 206, 208. During operation 706, the example first microcontroller 232 also causes the first sensor circuit 230 to continuously measure current across the first and second sealing electrodes to 206, 208. More specifically, during operation 704 the first sensor circuit 230 digitally samples sealer stage voltage values across the first and second sealing electrodes to 206, 208 during one or more sealing stage sampling time windows and performs a Fast Fourier Transform (FFT) to determine frequency content of the voltage across the first and second sealing electrodes to 206, 208. During operation 706 the first sensor circuit 230 digitally samples sealer stage current values across the first and second sealing electrodes to 206, 208 during the one or more sealing stage sampling time windows and performs an FFT to determine frequency content of the voltage across the first and second sealing electrodes to 206, 208. In an example sealing procedure, the digital sampling rate is in a range 1-10 milliseconds per sample. During arc detection operation 708, the example first micro-controller 232 is configured to continuously evaluate whether the frequency content in the measured RF sealer stage current waveform in combination with the measured RF sealer stage voltage waveform indicates an actual or imminent occurrence of an aberrant electrical arc event. An example first micro-controller 232 is configured to compare frequency content of the measured sealer stage current waveform to frequency content of the measured sealer stage voltage waveform to evaluate the extent of electrical non-linearity, if any, of the biological tissue load, and thereby to detect whether an aberrant arc occurs or is imminent. An alternate arc detection operation detects an occurrence of an arc event based only upon measured current content. During adjustment operation 710, the example first micro-controller 232 controls the first buck regulator 218 to adjust a control voltage based upon the arc detection determination. For example, in response to detection of an aberrant arc event during the detection operation 708, the adjustment operation 710 causes the first buck regulator 218 to output a control voltage to cause the first output transformer 220 to output a reduced energy level RF signal. In response to detection of no aberrant arc event during the detection operation 708, the adjustment operation 710 causes the first buck regulator 218 to output a control voltage to cause the first output transformer 220 to continue to output the predetermined clinical level energy RF signal for sealing.

Still referring to the example ESG 200 of FIG. 4, during an electrosurgical dissection procedure using the cutting stage 204, operation 702 the first microcontroller 262 involves control the energy level of an RF signal output by the second buck regulator 248 to produce a voltage control signal to cause the second output transformer 250 according to a clinical cutting procedure protocol. In the absence of an aberrant arc event, the second micro-controller 262 causes the second output transformer 250 to output an RF signal having a predetermined clinical energy level according to a predetermined clinical procedure protocol suitable to cut biological tissue. The predetermined clinical energy level includes current and voltage values selected to deliver energy to a biological tissue load coupled between the conductors 210, 212 that is sufficient to cut tissue without causing unwanted tissue damage and without causing unwanted damage to the instrument, for example. The predetermined clinical energy level may cause some acceptable level of arcing within a plasma layer that may be formed near the surface of a tissue to be cut. During operation 704, the example second microcontroller 262 causes the second sensor circuit 260 to continuously measure voltage across the first and second cutting electrode conductors to 210, 212. It is noted that in the example system 200, return electrodes 208, 212 (seal−, cut−) are shared in common. During operation 706, the example second microcontroller 262 also causes the second sensor circuit 260 also continuously measure current across the first and second cutting electrodes to 210, 212. More specifically, during operation 704 the second sensor circuit 260 digitally samples cutting stage voltage values across the first and second cutting electrodes to 210, 212 during one or more cutting stage sampling time windows and performs an FFT to determine frequency content of the voltage across the first and second cutting electrodes to 210, 212. During operation 706 the second sensor circuit 260 digitally samples cutting stage current values across the first and second sealing electrodes to 210, 212 during the one or more cutting stage sampling time windows and performs an FFT to determine frequency content of the voltage across the first and second cutting electrodes to 210, 212. In an example cutting procedure, the digital sampling rate is in a range 1-10 milliseconds per sample. During arc detection operation 708, the example second micro-controller 262 is configured to continuously evaluate whether the frequency content in the measured RF cutting stage current waveform in combination with the measured RF cutting stage voltage waveform indicates an actual or imminent occurrence of an aberrant electrical arc event. An example second micro-controller 262 is configured to compare frequency content of the measured cutting stage current waveform to frequency content of the measured cutting stage voltage waveform to evaluate the extent of electrical non-linearity, if any, of the biological tissue load, and thereby to detect whether an aberrant arc occurs or is imminent is occurring or is about to occur the other of the cutting electrodes 210, 212. An alternate arc detection operation detects an occurrence of an arc event based only upon measured current content. During adjustment operation 710, the example second micro-controller 262 controls the second buck regulator 248 to adjust a control voltage based upon the arc detection determination. For example, in response to detection of an aberrant arc event during the detection operation 708, the adjustment operation 710 causes the second buck regulator 248 to output a control voltage to cause the second output transformer 250 to output a reduced energy level of the RF signal. In response to detection of no aberrant arc event during the detection operation 708, the second buck regulator 248 outputs a control voltage to cause the second output transformer 250 to continue to output the predetermined clinical level energy RF signal for cutting.

E. Detecting an Arc Event Based Upon Frequency Content of Electrical Signals Measured Across a Biological Tissue Electrical Load

In the example ESG 200 (sealer stage 202 or dissection stage 204), of FIG. 4, the arc detection operation 708 evaluates electrical linearity of a biological tissue electrical load in both the sealer stage 202 and the dissection stage 204, to detect occurrences of an aberrant arc event, based upon the frequency content of one or more electrical signal waveforms measured across the electrical load.

A first example arc detection operation 708 evaluates total harmonic distortion (THD) of a waveform of an RF electrical current signal flowing through a biological tissue electrical load to evaluate whether there is an occurrence of an aberrant arc event. THD is a mathematical calculation that compares the output of higher order resonant frequencies in a signal to its fundamental frequency and that can be represented as percent deviation from a pure sine wave, with an increasing percentage as the signal becomes more deviated. More particularly, detection operation 708 evaluates THD by detecting magnitude of signal at the fundamental frequency and by determining magnitude of individual harmonics above the fundamental frequency. The signal magnitude content for the higher order harmonics is summed, typically up to the Nyquist sampling rate. The sum of the magnitudes of the higher order harmonics is compared with the magnitude of the original signal, to determine a THD value. Stated differently, operation 708 determines a THD of the current signal waveform as a ratio of noise introduced by arcing, measured in terms of the sum of magnitudes of higher order harmonics, divided by magnitude of fundamental frequency, the larger the value of THD, the higher the noise due to arcing. For example, a pure sine wave has a THD of 0%, a slightly distorted sine wave could have a THD of 10%, and a sine wave sufficiently distorted to become a square wave has a THD of about 50%. In an example ESG 200 in which the RF output signal is essentially sinusoidal, a simple evaluation of the THD of the RF current waveform could be adequate detect whether the energy delivery at the electrode was purely resistive, had some non-linearities as a result of the plasma discharge, or has significant non-linearities as a result of an arc discharge. During an arc event, the frequency content of current flow through biological tissue generally experiences more significant perturbation and corresponding harmonic distortion due to an arc event than does the frequency content of voltage across the biological tissue. Accordingly, the first example operation 708 evaluates presence of an arc event based only upon the measured current waveform. The first example detection operation 708 detects whether the THD of the electrical current signal waveform meets a threshold limit, for example, that is indicative on an aberrant arc event. For instance, a first example detection operation detects whether the THD of an electrical current signal wavefrom increases by ten per cent or greater from a baseline value for that waveform, which is indicative of an aberrant event. In such example instance, if the baseline value is 20, then the threshold for detection would be 22. Alternatively, the first example arc determination operation 708 can use signal to noise ratio (SNR) of the electrical current signal waveform instead of THD. SNR is the ratio of the signal frequency versus noise. Alternatively, the first example arc detection operation 708 can use signal to noise and distortion ratio (SINAD) of the electrical current signal waveform instead of THD. SINAD is the ratio of magnitude of signal frequency in RMS to the RMS of magntides of noise and/or harmonic frequencies.

A second example arc detection operation 708 is used with an alternative example ESG 200 that produces an RF output signal that is not sinusoidal. The second example arc detection operation determines a ratio of a THD_{I} of an electrical current signal waveform to a TDH_{V} of an electrical voltage signal waveform, which provides an indication of the non-linearity of the biological tissue load. More particularly, the ratio, THD_{I}/THD_{V}, provides an indication of the extent to which the electrical current signal waveform deviates from an electrical voltage signal waveform imparted by the ESG, due to non-linear component of the load. In general, nonlinearity of a tissue load due to an arc event has a greater impact upon frequency content of the measured current signal wavefrom than upon the measured voltage signal waveform. The second example detection operation 708 determines whether the THD_{I}/THD_{V}, meets a threshold limit of that is indicative on an aberrant arc event. For instance, a second example detection operation detects whether THD_{I}/THD_{V}>1.1. Alternatively, the second example arc determination operation 708 can evaluate whether a ratio of SNR_{I} of the electrical current signal waveform to SNR_{V} of the electrical voltage waveform (SNR_{I}/SNR_{V}) meets a threshold to detect an aberrant arc event. Alternatively, the second example arc detection operation 708 can evaluate whether a ratio of SINAD_{I} of the electrical current signal waveform to SINAD_{V} of the electrical voltage signal waveform (SINAD_{I}/SINAD_{V}) meets a threshold to detect an aberrant arc event.

The first and second example detection operations 708 can use specific frequency ranges above the fundamental frequency to determine values for THD, SNR, and SINAD. The noise caused by an arc event often occurs in a specific frequency range. Even in the absence of an arc event, some noise exists at some frequencies above the fundamental frequency in current and voltage signal waveforms measured across a tissue. Moreover, an arc event typically has a greater impact upon some frequencies than others. Including frequencies that are not as much influenced by unwanted arc events can dilute the effectiveness of THD, SNR or SINAD evaluations in detecting aberrant arc events. Thus, focusing THD, SNR or SINAD determinations on the frequency ranges most impacted by an arc event can provide a better indication of an occurrence of an arc event. In general, higher frequencies are less influenced by an arc event, and therefore, THD, SNR and SINAD determinations focus on lower frequency harmonics above the fundamental frequency. In an example arc determination operation 708, magnitude of one or more of the third through fifth harmonics above the fundamental are used in evaluating THD, SNR and/or SINAD to detect aberrant arc events.

A third example detection operation 708 uses a Q-factor (quality factor) to detect an occurrence of an aberrant arc event. A Q factor represents a ratio of a central frequency to the bandwidth around that frequency. A Q-factor often is used to determine the damping of a circuit. The high frequencies of an arc discharge generally increase the bandwidth of both the fundamental and resonant frequencies, which decreases the Q factor and making a resonator appear more overdamped. The calculated Q factor can be used in combination with or independently of THD, SNR, or SINAD or ratios involving THD, SNR, or SINAD, as described above, to determine an occurrence of an arc event.

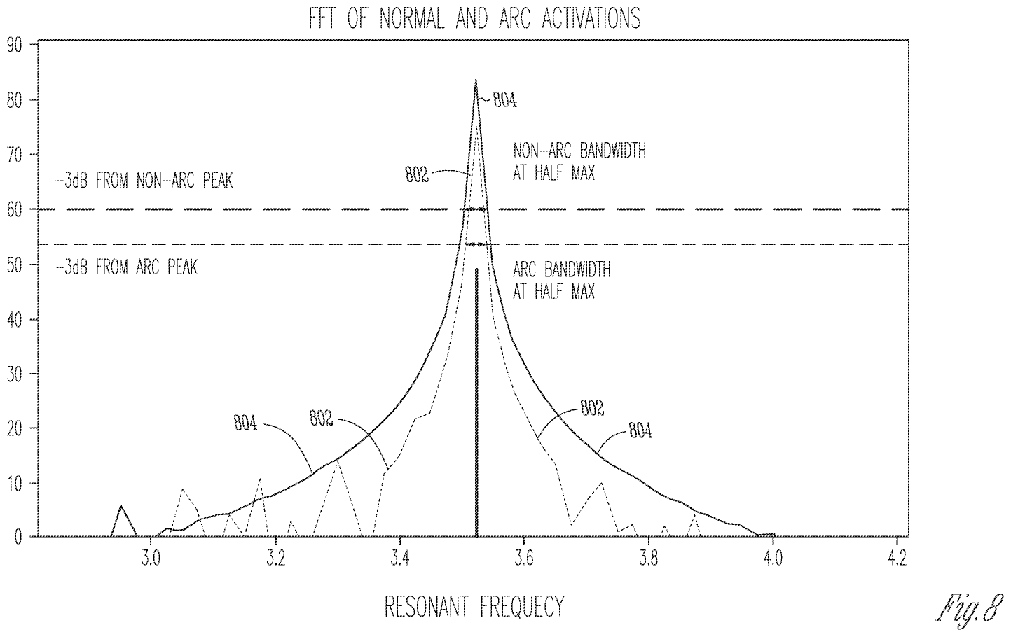

A third example detection operation 708 determines a Q-factor based upon an FFT of a measured RF signal waveform (current or voltage) based upon the equation: Q=resonant frequency/bandwidth at −3 dB from peak. Thus, a Q-factor looks at the fundamental frequency resonance of the system instead of the harmonic frequencies. A larger value of Q-factor is indicative of an occurrence of an arc event. In an example ESG 200, an increase in Q-factor value by ten per cent is indicative of an arc event. Thus, for example, the threshold would be a Q factor increasing from 20 to 22. FIG. 8 is an illustrative graph that shows a comparison between an FFT of a waveform influenced by an arc event arc 802 and a wavefrom not influenced by an arc 804 event, illustrating how the Q-is calculated for each. By way of explanation, it is believed that when activating into a real load (e.g., biological tissue) the system is damped and the amplitudes of higher order harmonic frequencies are low. As the frequency content of the load changes to contain real and imaginary components during an arcing event, the damping decreases, more ringing occurs, and the amplitude of higher order frequencies increases. A damped system during normal activations corresponds to a low Q-factor, and the Q-factor will increase as the damping decreases during an arc.

F. Control of RF Energy Delivery Based Upon Arc detection of an Occurrence of an Arc Event

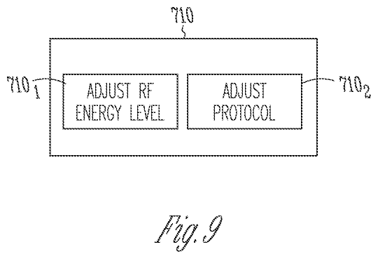

FIG. 9 is an illustrative flow diagram representing certain details of an example adjustment operation 710. An adjustment operation 710 includes an energy level adjustment operation 710₁ and protocol adjustment operation 710₂. The RF energy level adjustment operation 710₁ and the protocol adjustment operation 710₂ respectively adjust RF energy level and RF energy delivery protocol in response to information received from the detection operation 708.

An example energy level adjustment operation 710₁ adjusts the energy level delivered during a sealing or cutting procedure based upon information produced by the arc detection operation 708. For example, an example energy level adjustment operation 710₁ can be selectably configured to interrupt delivery of RF energy level in response to the detection operation 708 providing information indicating that an arc event that exceeds a prescribed threshold. An example energy level adjustment operation 710₁ can interrupt delivery for a prescribed time interval. An alternative example RF energy level adjustment operation 710₁ is selectably configured to adjust RF energy delivery level as a function of frequency content of a measured an electrical current or voltage waveform or combination of the two to maintain a frequency content within a range indicative of absence of an aberrant arc event. Another alternative example energy level adjustment operation 710₁ is configured to regulate RF energy level as a function of a selected high frequency harmonics of an electrical current signal waveform. For example, an example energy level adjustment operation 710₁ can be configured to regulate RF energy level to maintain a prescribed frequency range of the electrical current signal waveform selected to ensure adequate clinical performances such as sealing or cutting, and to exclude frequency content indicative of an occurrence of an aberrant arc. Yet another alternative example energy level adjustment operation 710₁ is configured to regulate RF energy level to maintain a prescribed value of the ratio of the current and voltage wave forms such as THD_{I}/THD_{V} SNR_{I}/SNR_{V} or SINAD_{I}/SINAD_{V}) selected to ensure adequate clinical performances such as sealing or cutting, and to exclude frequency content indicative of an occurrence of an aberrant arc. Still another alternative example energy level adjustment operation 710₁ is configured to regulate RF energy level to maintain a prescribed value for a Q-factor selected to ensure adequate clinical performances such as sealing or cutting, and to exclude frequency content indicative of an occurrence of an aberrant arc.

An example protocol adjustment operation 710₂ adjusts the clinical procedure protocol for an electrosurgery procedure during a sealing or cutting procedure based upon information produced by the arc detection operation 708. An example protocol adjustment operation 710₂ adjusts the clinical procedure protocol for an electrosurgery procedure as a function of the RF energy level delivered in response to detection of an aberrant arc event. For instance, an example sealing or cutting protocol may require a prescribed number of RF energy pulses at a prescribed RF energy level during a prescribed time interval. However, in response to information received from the arc detection operation indicating detection of an aberrant arc event, one or more of the energy pulses might be skipped during or may have a reduced energy level, during an occurrence of the aberrant arc event. An example protocol adjustment operation 710₂ adjusts the clinical procedure protocol for the procedure on-the-fly to make up for the missed or reduced-energy level pulses, to achieve desired clinical result such as sealing or cutting, despite the aberrant arc event. For example, a typical sealing or cutting operation involves a prescribed procedure-dependent number of pulses. Each typically has a duration in a range 100-200 milliseconds. For instance, in an example protocol adjustment operation 710₂, if an aberrant arc is detected during a scheduled delivery of one or more sealing or cutting pulses, those pulses may be skipped in response to the energy level adjustment operation 710₁ , but the protocol adjustment operation 710₂ may add one or more pulses to the sequence of pulses in the clinical procedure protocol to make up for the skipped one or more pulses.

Although illustrative examples have been shown and described, a wide range of modification, change and substitution is contemplated in the foregoing disclosure and in some instances, some features of the examples may be employed without a corresponding use of other features. One of ordinary skill in the art would recognize many variations, alternatives, and modifications. Thus, the scope of the disclosure should be limited only by the following claims, and it is appropriate that the claims be construed broadly and in a manner consistent with the scope of the embodiments disclosed herein. The above description is presented to enable any person skilled in the art to create and use electrosurgical signals to simultaneously seal and cut biological tissue. Various modifications to the examples will be readily apparent to those skilled in the art, and the generic principles defined herein may be applied to other embodiments and applications without departing from the spirit and scope of the invention. In the preceding description, numerous details are set forth for the purpose of explanation. For example, the ESG may include an FPGA circuit to evaluate frequency content of electrical signal waveforms at a target tissue and a separate processor circuit configured to adjust RF energy level and clinical procedure protocol. However, one of ordinary skill in the art will realize that the invention might be practiced without the use of these specific details. In other instances, well-known processes are shown in block diagram form in order not to obscure the description of the invention with unnecessary detail. Identical reference numerals may be used to represent different views of the same or similar item in different drawings. Thus, the foregoing description and drawings of embodiments in accordance with the present invention are merely illustrative of the principles of the invention. Therefore, it will be understood that various modifications can be made to the embodiments by those skilled in the art without departing from the spirit and scope of the invention, which is defined in the appended claims.

1. A method to control delivery of heat to biological tissue comprising:
imparting an RF electrical signal to the biological tissue over a circuit that includes a first electrode and a second electrode, wherein the biological tissue is electrically coupled between the first electrode and the second electrode;
measuring frequency content of the RF electrical signal between the first electrode and the second electrode; and
adjusting the RF electrical signal based upon the measured frequency content of the RF electrical signal.

2. The method of claim 1, wherein measuring frequency content of the RF electrical signal includes:
determining at least one of total harmonic distortion (THD), signal to noise ratio (SNR) and to noise and distortion ratio (SINAD) of an RF current signal component of the RF electrical signal, flowing between the first electrode and the second electrode.

3. The method of claim 1, wherein measuring frequency content of the RF electrical signal includes:
digitally sampling an RF current signal component of the RF electrical signal flowing between the first electrode and the second electrode the RF current signal; and
determining at least one of THD, SNR, and SINAD of the RF current signal component based upon digital samples of the RF current signal.

4. The method of claim 1, wherein measuring frequency content of the RF electrical signal includes:
determining at least one of a ratio of a respective THD, SNR, and SINAD of the RF current signal component of the RF electrical signal flowing between the first electrode and the second electrode to a respective THD, SNR, and SINAD of the RF voltage signal component of the RF electrical signal across the first electrode and the second electrode.

5. The method of claim 1, wherein measuring frequency content of the RF electrical signal includes,
digitally sampling an RF current signal component of the RF electrical signal flowing between the first electrode and the second electrode;
digitally sampling an RF voltage signal component of the RF electrical signal across the first electrode and the second electrode; and
determining at least one of a ratio of a respective THD, SNR, and SINAD of the RF current signal component to a respective THD, SNR, and SINAD of the RF voltage signal component, based upon digital samples of the RF current signal component and digital samples of the RF voltage signal component.

6. The method of claim 1, wherein measuring frequency content of the RF electrical signal includes,
determining a quality factor of the RF current component of the RF electrical signal flowing between the first electrode and the second electrode the RF current signal.

7. The method of claim 1, wherein adjusting the RF electrical signal based upon the frequency content of the RF electrical signal includes:
halting the RF electrical signal in response to a frequency content of the RF electrical signal meeting a threshold indicative of an occurrence of an aberrant arc.

8. The method of claim 1, wherein adjusting the RF electrical signal based upon the frequency content of the RF electrical signal includes:
adjusting energy level of the RF electrical signal to adjust frequency content of the RF electrical signal, based upon the determined frequency content of the RF electrical signal, to maintain frequency content of the RF electrical signal within a prescribed frequency range.

9. The method of claim 1, wherein adjusting the RF electrical signal based upon the frequency content of the RF electrical signal includes:
adjusting energy level of the RF electrical signal to adjust a voltage level of a voltage signal component of the RF electrical signal, based upon the determined frequency content of the RF electrical signal, to maintain frequency content of the RF electrical signal within a prescribed frequency range.

10. The method of claim 1,
wherein imparting the RF electrical signal includes imparting the RF electrical signal according to a predetermined protocol; and
wherein adjusting the RF electrical signal includes adjusting the predetermined protocol signal based upon the measured frequency content of the RF electrical signal.

11. The method of claim 1,
wherein imparting the RF electrical signal includes imparting the RF electrical signal according to a predetermined protocol that includes imparting a prescribed sequence of RF electrical signal pulses to the biological tissue; and
wherein adjusting the RF electrical signal includes adjusting the prescribed sequence of RF electrical signal pulses to the biological tissue.

12. An electrosurgical system to deliver heat to biological tissue comprising:
a radio frequency (RF) electrical signal source operable to impart an RF electrical signal to the biological tissue;
a digital sampling circuit coupled to digitally sample the RF electrical signal imparted to the biological tissue;
frequency content logic coupled to determine frequency content of the RF electrical signal based upon digital samples of the RF electrical signal; and
control logic coupled to control the RF electrical signal source based upon the determined frequency content of the RF electrical signal.

13. The electrosurgical system of claim 12,
wherein the digital sampling circuit includes one or more analog to digital converter circuits.

14. The electrosurgical system of claim 12,
wherein the frequency content logic determines at least one of total harmonic distortion (THD), signal to noise ratio (SNR) and to noise and distortion ratio (SINAD) of an RF current signal component of the RF electrical signal.

15. The electrosurgical system of claim 12,
wherein the digital sampling circuit includes one or more analog to digital converter circuits; and
wherein the frequency content logic determines at least one of THD, SNR, and SINAD of the RF current signal component based upon digital samples of the RF current signal.

16. The electrosurgical system of claim 12,
wherein the frequency content logic determines at least one of a ratio of a respective THD, SNR, and SINAD of the RF current signal component of the RF electrical signal to a respective THD, SNR, and SINAD of the RF voltage signal component of the RF electrical signal.

17. The electrosurgical system of claim 12,
wherein the digital sampling circuit includes one or more digital sampling circuits coupled to digitally sample an RF current signal component of the RF electrical signal;
wherein the digital sampling circuit includes one or more digital sampling circuits coupled to digitally sample an RF voltage signal component of the RF electrical signal; and
wherein the frequency content logic determines at least one of a ratio of a respective THD, SNR, and SINAD of the RF current signal component of the RF electrical signal to a respective THD, SNR, and SINAD of the RF voltage signal component of the RF electrical signal.

18. The electrosurgical system of claim 12,
wherein the frequency content logic determines a quality factor of the RF current component of the RF electrical signal.

19. The electrosurgical system of claim 12,
wherein the control logic is configured to cause the RF electrical signal source to halt imparting of the RF electrical signal in response to the determined frequency content of the RF electrical signal being indicative of an occurrence of an aberrant arc.

20. The electrosurgical system of claim 12,
wherein the control logic is configured to cause the RF electrical signal source to adjust an energy level of the RF electrical signal, based upon the determined frequency content of the RF electrical signal, to maintain frequency content of the RF electrical signal within a prescribed frequency range.

21. The electrosurgical system of claim 12,
wherein the control logic is configured to cause the RF electrical signal source to adjust a voltage level of a voltage signal component of the RF electrical signal, based upon the determined frequency content of the RF electrical signal, to maintain frequency content of the RF electrical signal within a prescribed frequency range.

22. The electrosurgical system of claim 12,
wherein the control logic is configured to cause the RF electrical signal source to impart the RF electrical signal according to a predetermined protocol and to adjust the predetermined protocol signal, based upon the determined frequency content of the RF electrical signal.

23. The electrosurgical system of claim 12,
wherein the control logic is configured to cause the RF electrical signal source to impart the RF electrical signal according to a predetermined protocol that includes imparting a prescribed sequence of RF electrical signal pulses to the biological tissue and to adjust the prescribed sequence of RF electrical signal pulses to the biological tissue, based upon the determined frequency content of the RF electrical signal.

24. The electrosurgical system of claim 12,
wherein the RF electrical signal source includes a transformer circuit.

25. The electrosurgical system of claim 12,
wherein the RF electrical signal source includes a transformer circuit configured to impart the RF electrical signal and including a voltage control circuit coupled to control a voltage level of the RF electrical signal imparted by the transformer circuit.

26. The electrosurgical system of claim 12,
wherein the frequency content logic includes processor circuitry configured according to executable instructions to determine the frequency content of the RF electrical signal based upon digital samples of the RF electrical signal; and
control logic includes the processor configured according to executable instructions to control the RF electrical signal source based upon the determined frequency content of the RF electrical signal.

27. The electrosurgical system of claim 12,
wherein the frequency content logic includes Field Programmable Gate Array circuitry configured to determine the frequency content of the RF electrical signal based upon digital samples of the RF electrical signal; and
control logic includes processor circuitry configured according to executable instructions to control the RF electrical signal source based upon the determined frequency content of the RF electrical signal.

28. The electrosurgical system of claim 12,
wherein the RF signal source includes a first electrode and a second electrode that are configurable to electrically couple the biological tissue therebetween; and
wherein the RF signal source is operable to impart the RF electrical signal over the first electrode and the second electrode with the biological tissue electrically coupled therebetween.
